(19) United States
(12) Patent Application Publication
(10) Pub. No.: US 2016/0135625 A1
(43) Pub. Date: May 19, 2016

(54) HEAD REST FOR SUPPORTING A PERSON'S HEAD WHEN RESTING IN A CHAIR

(71) Applicant: Vincenzo Cante, Newport Beach, CA (US)

(72) Inventor: Vincenzo Cante, Newport Beach, CA (US)

(21) Appl. No.: 14/546,891

(22) Filed: Nov. 18, 2014

(51) Int. Cl. A47G 9/10; A61F 9/04

(52) U.S. Cl. CPC A47G 9/1045; A47G 9/10; A61F 9/04; A47G 2009/003

(57) ABSTRACT

A combination headrest and eyeshade is disclosed. The headrest provides left and right lateral support while the eyeshade provides forward support so that the user's head does not tilt forward or to the sides when resting in a chair. The eyeshade is attached to a rear portion of the headrest so that any forward pressure applied thereto by the person's head is applied to the eyeshade and transmitted to the rear portion of the headrest through straps attached to the eyeshade and the headrest to prevent the person's head from falling forward.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The embodiments disclosed herein relate to a headrest and eyeshade to help a person sleep in a chair.

The United States Department of Transportation's Bureau of transportation statistics reported in 2003 that 815.3 million scheduled passengers traveled on U.S. airlines and foreign airlines in the United States in 2012. This was an increase of 1.3% from 2011. Some of these passengers prefer to sleep or rest their eyes while flying. As a result, products have come onto the marketplace to help passengers maintain the person's head in an erect position so that the person does not develop a neck cramp while sleeping. Unfortunately, these prior art devices have certain deficiencies that prevent the user from sleeping comfortably in an airplane chair.

Accordingly, there is a need in the art for an improved device to aid a passenger to sleep or rest in a seat.

### BRIEF SUMMARY

The various embodiments and aspects disclosed herein address the needs discussed above, discussed below and those that are known in the art.

A combination headrest and eye covering 22 is disclosed herein. The headrest provides left and right lateral support to the wearer's head so that the wearer can sleep while sitting down in a chair. The eye covering 22 is secured to a rear portion 28 of the headrest so that the eye covering 22 can support the head of the person from falling forward while sleeping in the chair. Accordingly, the combination headrest and eye covering provides both lateral as well as forward support so that the person can comfortably sleep in a chair without developing a neck cramp.

More particularly, a head support for providing lateral and forward support to a head of a person while the person is resting in a chair is disclosed. The head support may comprise a pillow, first and second straps and an eyeshade. The pillow may be disposed about a neck of the person. The pillow may be sufficiently large (e.g., thick) to provide lateral support to the head of the person when resting in the chair. The first and second straps may be attached to a rear portion of the pillow. The first and second straps may be disposable about left and right sides of the head of the person. The eyeshade may be attached to the first and second straps. In this regard, the first and second straps may be attached to opposed end portions of the eyeshade. The eyeshade may be disposed on a forehead or over eyes of the person to provide forward support to the head of the person when resting in the chair.

The pillow may be curved (e.g., semi circular) to fit around the neck of the person. The pillow may be filled with fill material selected from a group consisting of microbeads, foam, polyfil, air, polystyrene, polyester pellets or combinations thereof. The first and second straps may each have a buckle for adjusting a length of the strap.

The first and second straps may be elastic so that lengths of the straps are adjustable so that the eyeshade is adjustable on the person's head. The eye support may be deployed over the eyes of the person and traverable to a stowed position so that the eye shade is displaced away from the eyes of the person.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of the headrest and eye covering for providing left and right lateral support and forward support to a person's head;

FIG. 2 is a top view of the headrest and eye covering shown in FIG. 1; and

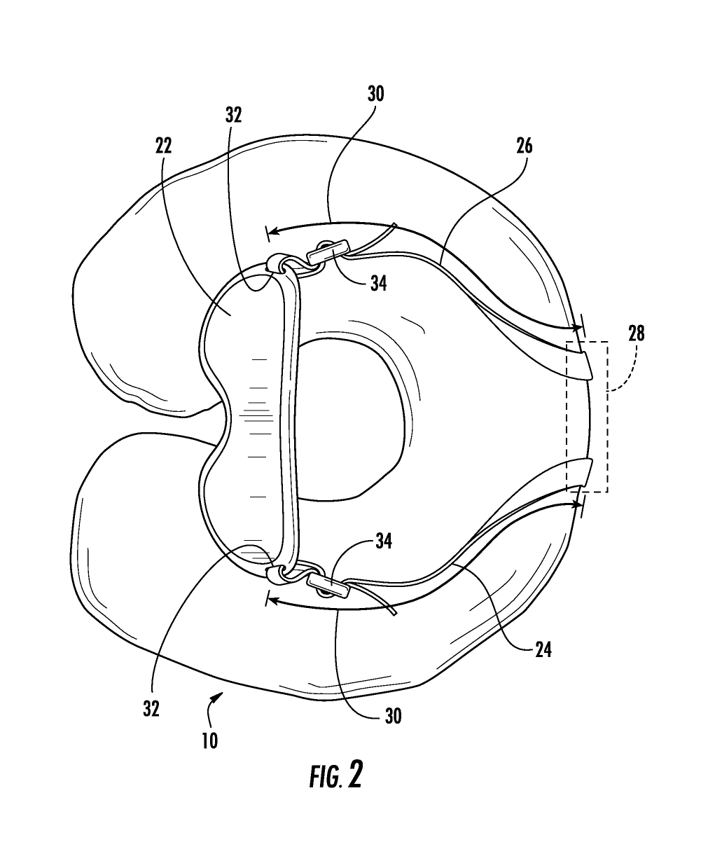

FIG. 3 is a side view of the headrest and eye covering having an optional flap.

### DETAILED DESCRIPTION

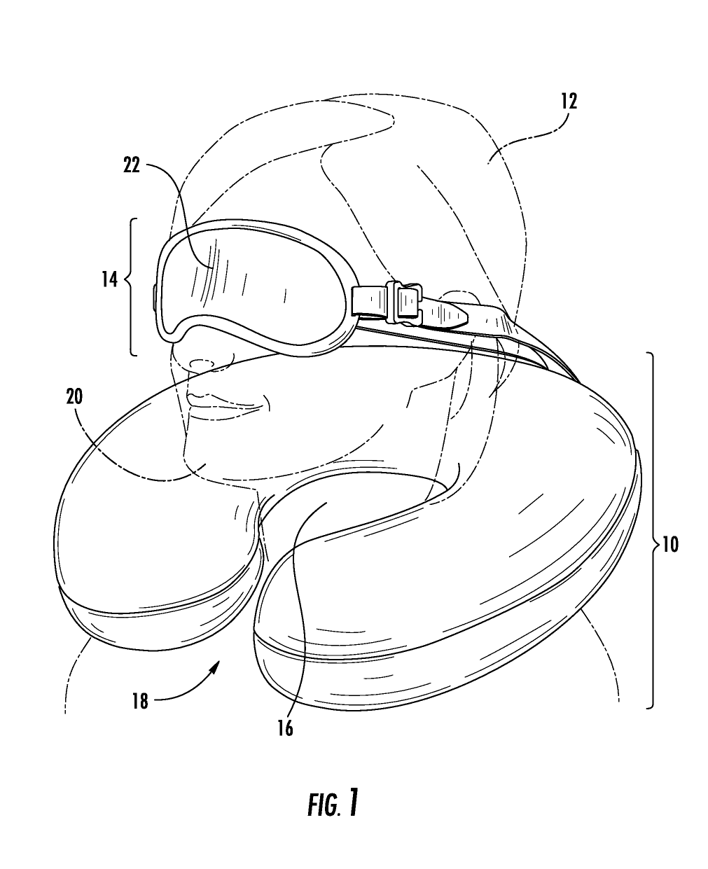

Referring now to the drawings, a headrest 10 for laterally supporting a person's head 12 in an erect position (see FIG. 1) and an eyeshade 14 for covering the person's eyes and supporting the person's head 12 to the rear position is shown. The eyeshade 14 is secured to a backside of the headrest 10 and is adjustable to cinch down the eyeshade 14 against the face of the person to prevent the person's head from falling forward while sleeping.

More particularly, referring now to FIG. 1, the headrest 10 has an enlarged C-shaped configuration which is disposable about a neck 16 of the person. The headrest 10 rests on top of the person's shoulders and supports the person's head 12 in the lateral left and right directions. In this regard, the person's head is maintained in an erect position, as shown in FIG. 1 to allow the person to sleep more comfortably while sitting down in a chair. These headrests 10 are typically used by passengers of an airline so that the passenger can sleep while flying. However, it is also contemplated that the headrest 10 may be for other purposes including but not limited to sleeping in a chair at home or other locations. The headrest 10 may be fabricated from a cloth material and be filled with fill material (e.g., cotton, buckweet, etc.). It is also contemplated that the headrest 10 may be fabricated from a plastic material and be inflatable with air. However, it is preferable that the headrest 10 be filled with fill material in order to provide weight and resistance so that the eyeshade 14 can hold the person's head 12 back and prevent the person's head from falling forward.

As discussed above, the headrest 10 may have a C-shaped configuration. This C-shaped configuration of the headrest 10 allows the person to slip the headrest 10 around the person's neck and to also take off the headrest 10 quickly and easily. The opening 18 is usually positioned in front of the wearer's face so that the chin 20 of the person is aligned to the opening 18. In this sense, the headrest 18 provides no forward support to the person's head. As such, the headrest 10 does not directly physically push the person's head backward.

As discussed above, the headrest 10 is typically filled with fill material such as microbeads, foam, polyphenol, polystyrene, polyester pellets or combinations thereof. Preferably, the weight of the aggregate fill material may be about 1 to 5 pounds so that the eyeshade 14 can hold the person's head toward the back position and prevent the person's head from falling forward.

The eyeshade 14 may comprise an eye covering 22 and left and right straps 24, 26. The left and right straps may be fixedly secured to a rear portion 28 of the headrest 10. By way of example and not limitation, the straps 24, 26 may be stitched, sonically welded, glued or otherwise attached to the rear portion 28 of the headrest 10. The straps 24, 26 may be flexible but non-elastic so that the eyeshade 14 can support the person's head 12 to the rear. The left and right straps 24 may be adjustably secured to left and right sides of the eye covering 22 so that a length 30 of the left and right straps 24, 26 may be adjusted so that the eye covering 22 can place a comfortable amount of pressure against the person's face to hold the person's head back in a comfortable manner and also to position the forward most position of the person's head. In particular, the left and right straps are fed through an opening 32 in left and right sides of the eye covering 22. The straps 24, 26 are fed through a buckle 34 which provides a frictional holding system for the strap 24, 26 after the lengths 30 of the straps 24, 26 have been adjusted.

The eye covering 22 may be fabricated from a nontransparent material so that ambient light is not shown through the eye covering 22. This allows the person to experience complete darkness when the eye covering 22 is disposed over the person's eyes. However, it is also contemplated that the eye covering 22 may be fabricated from other types of material that may allow sunlight to penetrate through the eye covering 22. By way of example and not limitation, the eye covering 22 may be fabricated from a nontransparent polymer material covered with fabric to provide comfort to the person when disposed over the person's eyes. The opposed end portions of the polymer material may have the openings 32 for threading the straps 24, 26 through the eye covering 22. The interior side of the eye covering 22 may be plush to provide additional comfort to the person wearing the eye covering 22.

In use, the person may dispose the headrest 10 about the person's neck 16. The eyeshade 14 is brought in front of the person's eyes and the length of the left and right straps 24 are adjusted to provide proper pressure to the person's face and to properly position the person's head. To adjust the length of the straps 24, 26, the user threads the straps 24, 26 through the buckle 34 to shorten or lengthen the length of the straps 24, 26. It is also contemplated that the eyeshade 14 may be positioned over the person's forehead in the event that the person does not want the eyeshade 14 to cover the person's eyes.

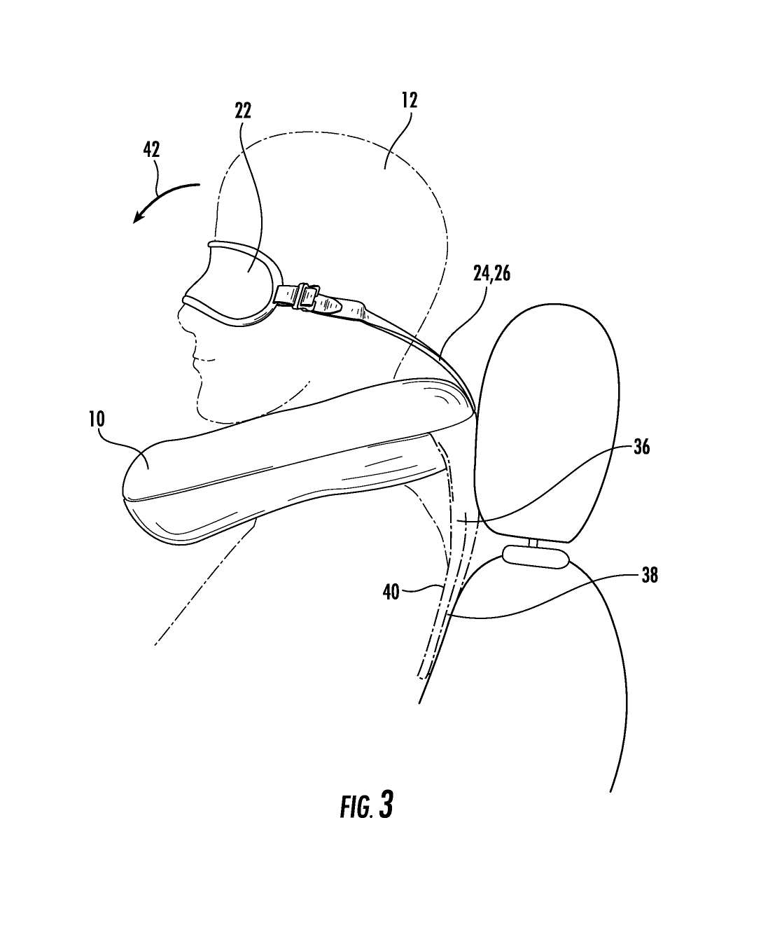

Referring now to FIG. 3, an alternate embodiment of the combination headrest 10 and eyeshade 14 is shown. The headrest has an additional flap 36 that extends downwardly and between the backrest 38 of the seat and a back 40 of the person so that the flap 36 can be gripped between the back 40 and the backrest 38. When the user's head 12 wants to fall forward in the direction of arrow 42, the force is transmitted through the eye covering 22, the left and right straps 24, 26 to the flap 36 which is held between the back 40 and the backrest 38.

The above description is given by way of example, and not limitation. Given the above disclosure, one skilled in the art could devise variations that are within the scope and spirit of the invention disclosed herein, including various ways of forming the head rest 10 and eyeshade 14. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A head support for providing lateral and forward support to a head of a person while the person is resting in a chair, the head support comprising:
a pillow disposable about a neck of the person, the pillow being sufficiently large to provide lateral support to the head of the person when resting in the chair;
first and second straps attached to a rear portion of the pillow, the first and second straps being disposable about left and right sides of the head of the person;
an eyeshade with the first and second straps attached to opposed end portions of the eyeshade, the eyeshade being disposable on a forehead or over eyes of the person to provide forward support to the head of the person when resting in the chair.

2. The head support of claim 1 wherein the pillow is curved to fit around the neck of the person.

3. The head support of claim 1 wherein the pillow is filled with fill material selected from a group consisting of microbeads, foam, polyfil, air, polystyrene, polyester pellets or combinations thereof.

4. The head support of claim 1 wherein the first and second straps each have a buckle for adjusting a length of the strap.

5. The head support of claim 1 wherein the first and second straps are elastic so that lengths of the straps are adjustable so that the eyeshade is adjustable on the person's head.

6. The head support of claim 6 wherein the eye support is deployable over the eyes of the person and traverable to a stowed away position so that the eye shade is displaced away from the eyes of the person.

7. The head support of claim 1 wherein the first and second straps are fabricated from a unitary elongate member, a middle portion of the unitary elongate member being attached to the rear portion of the pillow.
